(19) United States
(12) Patent Application Publication
(10) Pub. No.: US 2020/0376289 A1
(43) Pub. Date: Dec. 3, 2020

(54) IMPLANTABLE LIGHT THERAPY DEVICE

(71) Applicants: Nam Gyun Kim, Seoul (KR); Color Seven Co., Ltd., Seoul (KR)

(72) Inventors: Nam Gyun KIM, Seoul (KR); Kyoung Jun Park, Seoul (KR)

(21) Appl. No.: 16/425,036

(22) Filed: May 29, 2019

(51) Int. Cl. A61N 5/06

(52) U.S. Cl. CPC A61N 5/0601; A61B 5/0031; A61N 2005/0652; A61N 5/0613

(57) ABSTRACT

The present invention relates to an implantable light therapy device which emits visible light in a specific wavelength band for a predetermined time period according to symptoms of a disease in a state in which the implantable light therapy device is fixed to a disease site, which occurs in tissue or an organ in the human body due to inflammation to suppress or reduce inflammation inducers (for example, interleukin 1 beta (IL-1β) and interleukin 18 (IL-18)). When the inflammation inducers (for example, IL-β and IL-18) in a disease site generated due to the inflammation in the tissue or the organ of the human body are suppressed or reduced according to the present invention, inflammatory diseases (for example, Alzheimer's disease, Parkinson's disease, stroke, pancreatitis, rheumatoid arthritis) may be treated or prevented.

### BACKGROUND

### 1. Field of the Invention

The present invention relates to a light therapy device, and more specifically, to an implantable light therapy device for suppressing an inflammation inducer.

### 2. Discussion of Related Art

Since various papers have recently revealed that Alzheimer's disease, rheumatoid arthritis, pancreatitis, gout, and the like all occur through the same immunity mechanism, a possibility of a universal new drug development can be carefully checked.

All of the above-described three diseases are alike in that they are autoimmune diseases in which an immune body attacks itself and causes an immune response, and such autoimmune diseases are inflammatory diseases which occur due to excessive generation of a strong inflammation inducer that is cytokine interleukin-1β known as cytokines that are proteins secreted by immunocytes.

However, the secret of how and why interleukin-1β is excessively generated has been recently discovered.

When immune meditated macrophages, which find and digest foreign substances entering a body, feed on uric acid, protein complexes called nucleotide-binding oligomerization domain-like receptors family pyrin domain containing 3 protein (NLRP3) inflammasomes (which is a portmanteau word of inflammation and—some meaning a group) are generated in the macrophages. The NLRP3 inflammasome activates an enzyme called caspase-1 to generate the inflammation inducer interleukin-1β.

Surprisingly, it is reported that the NLRP3 inflammasome performs the same action in Alzheimer's disease. It means that β-amyloid, which is known as a cause of Alzheimer's disease, activates the inflammasome.

In addition, although substances which are causes of rheumatoid arthritis are not known yet, it is reported that the inflammasome plays an important role in generating rheumatoid arthritis.

As described above, due to the excessive generation of the inflammatory inducer interleukin-1β caused by the inflammasome, an inflammatory response occurs in each part of the body and the autoimmune disease occurs.

As a result, the inflammasome is activated by different danger signals at the cerebral cortex in the case of Alzheimer's disease, at a knee joint in the case of rheumatoid arthritis, and at a toe joint in the case of gout. In addition, an inflammatory response due to the inflammation inducer interleukin 1 beta (IL-1β) caused by the inflammasome causes immunocytes in the body to attack cells of inflamed parts. As described above, when the knee joint, the toe joint, pancreas, the cerebral cortex are attacked, different autoimmune diseases occur.

Accordingly, when a drug or apparatus capable of effectively suppressing the NLRP3 inflammasome is developed, the above-described autoimmune diseases can be easily cured or prevented.

However, a safe and effective drug which suppresses the NLRP3 inflammasome has not been developed yet.

Recently, it is reported that, when visible light with a specific wavelength is directly emitted on skin, cyclooxygenase-2 (COX-2) or the NLRP3 inflammasome is suppressed and thus generation of the inflammation inducer IL-162 can be suppressed.

### PRIOR ART DOCUMENT

### Patent Document

(Patent Document 1) Korean Patent Registration No. 10-1217176 B1

### SUMMARY OF THE INVENTION

The present invention is directed to providing an implantable light therapy device which emits visible light in a specific wavelength band for a predetermined time period according to symptoms of a disease in a state in which the implantable light therapy device is fixed to a disease site, which occurs in tissue or an organ in a human body due to inflammation, to suppress a priming process due to cyclooxygenase-2 (COX-2), toll-like receptor 2 (TLR-2), mitogen-activated protein kinase (MAPK), and a nuclear factor kappa-light-chain-enhancer of activated B cells (NK-kB) and to suppress an inflammasome activation process by suppressing nucleotide-binding oligomerization domain-like receptors family pyrin domain containing 3 protein (NLRP3) so that inflammation inducers (for example, interleukin 1 beta (IL-1β) and interleukin 18 (IL-18)) are suppressed or reduced.

According to an aspect of the present invention, there is provided an implantable light therapy device including: a light-emitting electrode housing which is configured to be implanted in a human body to suppress inflammation inducers, which includes a sealed inner space in which a printed circuit board (PCB) on which a light source required to emit light is installed is embedded therein, and which causes the light emitted from the light source to be emitted to a disease site due to inflammation of tissue or an organ of the human body, and in which a sealed transparent light outlet is installed toward an outside of the sealed inner space, and one or more holes configured to be fixed to the human body and a connecting terminal connected to an electric wire electrically connected to a light-emitting electrode connecting terminal installed in a body housing are installed on one outer surface of the sealed inner space; a body housing which is configured to be implanted in the human body and includes a sealed inner space in which a light-emitting controller, a battery for a power supply, and a solar cell configured to charge the battery, which are required to emit the light, are embedded, and in which a sealed transparent window is installed on one surface of the sealed inner space such that light externally enters the solar cell, and a light-emitting electrode connecting terminal connected to the light-emitting electrode housing is installed on one outer surface of the sealed inner space; and a remote controller configured to control light emission through wireless communication with the body housing from an outside of the human body.

The light source of the implantable light therapy device may be one or more of light-emitting diodes (LED), a micro LED array, or an organic LED (OLED).

The light source of the light-emitting electrode housing may emit light with a peak wavelength ranging from 590 nm to 630 nm and an intensity ranging from 1 mW/cm² to 10 mW/cm² to a disease site.

In the implantable light therapy device, the body housing may wirelessly communicate with the remote controller using any one among radio frequency (RF), Bluetooth low energy (BLE), Zigbee, near field communication (NFC) methods.

In the implantable light therapy device, a light-emitting control device of the body housing may include the light-emitting controller, a wireless transceiver, a storage memory, and a charge control circuit.

The remote controller may include a wireless transceiver, a light-emitting controller, and a battery, which are embedded therein, to wirelessly control light emission, and a touch-type display installed on one outer surface thereof to wirelessly control light emission to be turned on or off through the wireless transceiver disposed in the body housing and may have functions of wirelessly controlling any one or more of a wavelength of light, an intensity thereof, and a light-emitting time period and transmitting a user's light treatment result to a medical institution.

In the implantable light therapy device, the remote controller may have any one or more of functions of monitoring a remaining amount of charge of the battery embedded in the body housing, checking and storing a user's light treatment use history, and providing information about a light treatment method and a precaution to a user.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view illustrating a configuration of an implantable light therapy device according to an embodiment of the present invention;

FIG. 2 is a view illustrating configurations of a light-emitting electrode housing and a body housing of FIG. 1 according to the embodiment; and

FIG. 3 is a view illustrating a state in which the implantable light therapy device according to the present invention is implanted in a human body and directly attached to a pancreas.

### DETAILED DESCRIPTION OF EXEMPLARY EMBODIMENTS

Hereinafter, an exemplary embodiment of the present invention will be described in more detail with reference to the accompanying drawings.

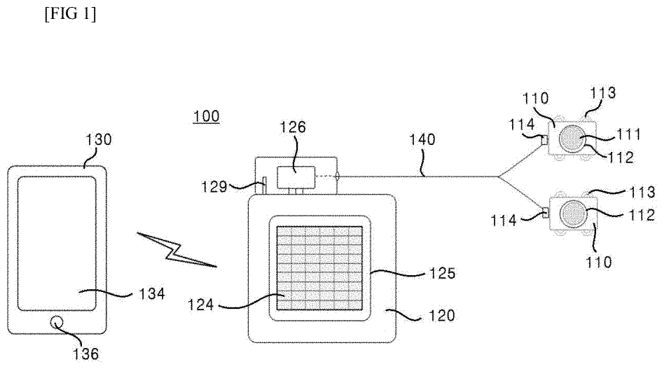

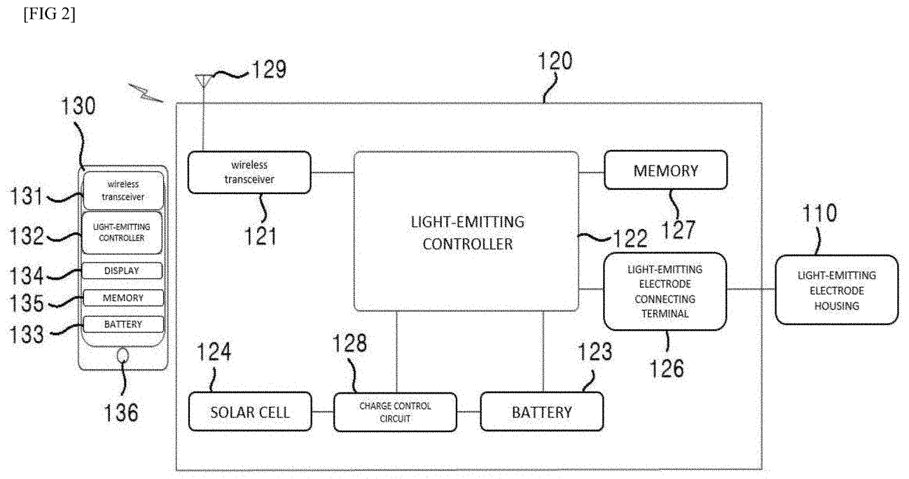

Referring to FIGS. 1 and 2, an implantable light therapy device 100 according to the present invention includes light-emitting electrode housings 110, a body housing 120, and a remote controller 130.

The light-emitting electrode housing 110 and the body housing 120 are implanted and installed in a human body and connected by an electric wire 140.

The light-emitting electrode housing 110 and the body housing 120 may be formed of materials having high biocompatibility and durability in the human body.

The body housing 120 may communicate with the remote controller 130 using any one among radio frequency (RF), Bluetooth low energy (BLE), Zigbee, and near field communication (NFC) methods.

The light-emitting electrode housing 110 is configured to be implanted in the human body and include a sealed inner space in which printed circuit board (PCB) is embedded, wherein a light source 111 required for emitting light is installed on the PCB so that light emitted by the light source 111 is emitted to a disease site occurring in tissue or an organ of the human body due to inflammation.

The light source 111 may be one or more light-emitting diodes (LEDs), a micro LED array, or an organic LED (OLED).

The light source 111 of the light-emitting electrode housing 110 emits light with a peak wavelength ranging from 590 nm to 630 nm and an intensity ranging from 1 mW/cm² to 10 mW/cm² to the disease site.

Light outlets 112, which are transparent and sealed, are installed in the light-emitting electrode housings 110 toward the outside of the sealed inner spaces.

One or more holes 113 configured to fix a human body and installed in one outer surface of the sealed inner space and a connecting terminal 114, which is connected to the electric wire 140 electrically connected to a light-emitting electrode connecting terminal 126 installed in the body housing 120, are installed in the light-emitting electrode housing 110.

The body housing 120 is configured to be implanted in the human body, and a sealed inner space is provided so that a wireless transceiver 121, a light-emitting controller 122, a battery 123 for a power supply, and a battery charging solar cell 124, which are required for emitting light, are embedded in the body housing 120. In addition, a sealed transparent window 125 is installed on one surface of the sealed inner space such that light externally enters the solar cell 124.

The light-emitting controller 122 of the body housing 120 may include a micro control unit (MCU).

The light-emitting electrode connecting terminal 126 which is connected to the light-emitting electrode housing 110 is installed on one outer surface of the sealed inner space of the body housing 120.

One or more light-emitting electrode connecting terminals 126 may be installed.

For example, one light-emitting electrode connecting terminal 126 may be connected to the plurality of light-emitting electrode housings 110, or the plurality of light-emitting electrode connecting terminals 126 may also be connected to correspond to the plurality of light-emitting electrode housings 110.

The body housing 120 further includes a memory 127, a charge control circuit 128, and an antenna 129.

Information about a remaining amount of charge of the battery 123, information about a user's light treatment use history, information about a light treatment method and precautions, and the like are stored in the memory 127 of the body housing 120.

When light reaches the solar cell 124 through the sealed transparent window 125, the charge control circuit 128 charges the embedded battery 123 for a power supply.

Wireless communication is performed between the body housing 120 and the remote controller 130 via the antenna 129.

The remote controller 130 wirelessly communicates with the body housing 120 from the outside of the human body so as to control light emission.

A wireless transceiver 131, a light-emitting controller 132, and a battery 133 are embedded in the remote controller 130 to wirelessly control light emission, and a touch-type display 134 is installed on one outer surface of the remote controller 130 to wirelessly control the light emission to be turned on or off through the wireless transceiver 121 disposed in the body housing 120.

The light-emitting controller 132 of the remote controller 130 may include an MCU.

The remote controller 130 further includes a memory 135 and an on/off switch 136 installed on an outer surface thereof.

Information about a remaining amount of charge of the battery 123 embedded in the body housing 120, information about a user's light treatment use history, information about a light treatment method and precautions, information about medical institution contacts to transmit a user's light treatment result (for example, phone numbers and email addresses), and the like are stored in the memory 135 of the remote controller 130.

The remote controller 130 has functions of wirelessly controlling any one or more among a wavelength of light, an intensity thereof, and a light-emitting time period and transmitting a user's light treatment result to the medical institution.

The remote controller 130 has any one or more of functions of monitoring the remaining amount of charge of the battery 123 embedded in the body housing 120, checking and storing the user's light treatment use history, and providing the information about the light treatment method and precautions to the user.

Any device capable of wirelessly communicating with the body housing 120 is used as the remote controller 130 without limitation, and the remote controller 130 may be a smart communication device such as a smart phone, an iPhone, an Android Phone, and a smart watch.

The implantable light therapy device 100 according to the present invention configured as described above is used as described below.

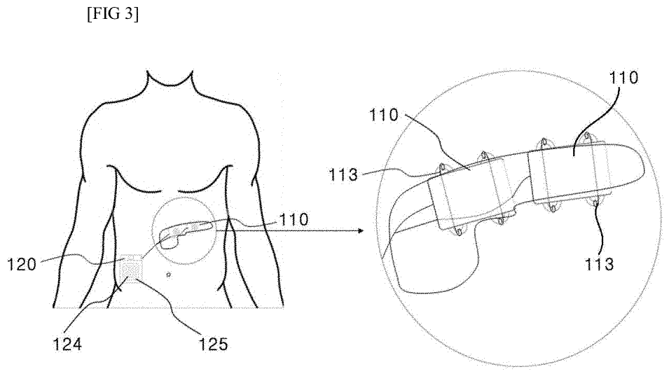

FIG. 3 is a view illustrating a state in which the implantable light therapy device 100 according to the present invention is implanted in the human body and directly attached to a pancreas.

As illustrated in FIG. 3, in a case in which the implantable light therapy device 100 according to the present invention is used to treat pancreatitis or pancreatic cancer, the body housing 120 is implanted in a space in flank skin of the human body, the light-emitting electrode housings 110 are attached to a disease site of the pancreas using the holes 113 configured to fix the human body and installed in the light-emitting electrode housings 110 configured to emit light, and light is emitted using the remote controller 130.

In this case, when the visible light in a specific wavelength band is emitted for a predetermined time period according to a symptom of the pancreatitis or the pancreatic cancer, a priming process due to cyclooxygenase-2 (COX-2), toll-like receptor 2 (TLR-2), mitogen-activated protein kinase (MAPK), and a nuclear factor kappa-light-chain-enhancer of activated B cells (NK-kB) is suppressed and an inflammasome activation process is suppressed by suppressing nucleotide-binding oligomerization domain-like receptors family pyrin domain containing 3 protein (NLRP3) and thus inflammation inducers (for example, interleukin 1 beta (IL-1β) and interleukin 18 (IL-18)) may be suppressed or reduced in the human body.

Particularly, while the implantable light therapy device 100 according to the present invention is implanted and used in the human body, when the battery 123 for a power supply embedded in the body housing 120 needs to be charged as a result of checking information about a remaining amount of charge of the embedded battery 123 for a power supply and light configured to charge is emitted from a skin surface of the human body toward the solar cell 124, the light configured to charge reaches the solar cell 124 through the sealed transparent window 125 installed on the body housing 120 implanted under the skin. When the light configured to charge reaches the solar cell 124, the embedded battery 123 for a power supply is charged by the charge control circuit 128.

As described above, when inflammation inducers (for example, IL-1β and IL-18) in a disease site generated due to inflammation in tissue or an organ of the human body are suppressed or reduced according to the present invention, inflammatory diseases (for example, Alzheimer's disease, Parkinson's disease, stroke, pancreatitis, rheumatoid arthritis) can be treated or prevented.

The above-described implantable light therapy device according to the present invention is not limited to the above-described embodiment, and the technical spirit covers a range in which the embodiment may be variously modified and practiced by those skilled in the art without departing from the gist of the present invention defined by the appended claims.

What is claimed is:

1. An implantable light therapy device comprising:
a light-emitting electrode housing which is configured to be implanted in a human body to suppress inflammation inducers, which includes a sealed inner space in which a printed circuit board (PCB) on which a light source required to emit light is installed is embedded therein, and which causes light emitted from the light source to be emitted to a disease site due to inflammation of tissue or an organ of the human body, and in which a sealed transparent light outlet is installed toward an outside of the sealed inner space, and one or more holes configured to fix the human body and a connecting terminal connected to an electric wire electrically connected to a light-emitting electrode connecting terminal installed in a body housing are installed on one outer surface of the sealed inner space;
a body housing which is configured to be implanted in the human body and includes a sealed inner space in which a wireless transceiver, a light-emitting controller, a battery for a power supply, and a solar cell configured to charge the battery, which are required to emit light, are embedded, and in which a sealed transparent window is installed on one surface of the sealed inner space such that light externally enters the solar cell, and a light-emitting electrode connecting terminal connected to the light-emitting electrode housing is installed on one outer surface of the sealed inner space; and
a remote controller configured to control light emission through wireless communication with the body housing from an outside of the human body.

2. The implantable light therapy device of claim 1, wherein a method of suppressing the inflammation inducers suppresses a priming process due to cyclooxygenase-2 (COX-2), toll-like receptor 2 (TLR-2), mitogen-activated protein kinase (MAPK), and a nuclear factor kappa-light-chain-enhancer of activated B cells (NK-kB) and also suppresses an inflammasome activation process by suppressing nucleotide-binding oligomerization domain-like receptors family pyrin domain containing 3 protein (NLRP3), and thus inflammation inducers (for example, interleukin 1 beta (IL-1β) and interleukin 18 (IL-18)) are suppressed or reduced.

3. The implantable light therapy device of claim 1, wherein the light source of the light-emitting electrode housing emits light, which has a peak wavelength ranging from 590 nm to 630 nm and an intensity ranging from 1 mW/cm² to 10 mW/cm², to a disease site.

4. The implantable light therapy device of claim 1, wherein the body housing wirelessly communicates with the remote controller.

5. The implantable light therapy device of claim 1, wherein the body housing further includes a memory and a charge control circuit.

6. The implantable light therapy device of claim 1, wherein the remote controller:
includes a wireless transceiver, a light-emitting controller, and a battery, which are embedded therein, to wirelessly control light emission, and a touch-type display installed on one outer surface thereof to wirelessly control light emission to be turned on or off through the wireless transceiver disposed in the body housing; and
has functions of wirelessly controlling any one or more of a wavelength of light, an intensity thereof, and a light-emitting time period and transmitting a user's light treatment result to a medical institution.

7. The implantable light therapy device of claim 1, wherein the remote controller has any one or more of functions of monitoring a remaining amount of charge of the battery embedded in the body housing, checking and storing a user's light treatment use history, and providing information about a light treatment method and a precaution to a user.
